(19) United States
(12) Patent Application Publication
(10) Pub. No.: US 2023/0225674 A1
(43) Pub. Date: Jul. 20, 2023

(54) SENSOR-BASED GARMENT FOR MONITORING OF BREAST MILK PRODUCTION

(71) Applicant: Lilu Inc., New York, NY (US)

(72) Inventors: Adriana Catalina Vazquez Ortiz, New York, NY (US); Sujay Suresh Kumar, New York, NY (US)

(21) Appl. No.: 17/900,514

(22) Filed: Aug. 31, 2022

Related U.S. Application Data
(60) Provisional application No. 63/300,781, filed on Jan. 19, 2022.

(51) Int. Cl. A61B 5/00

(52) U.S. Cl. CPC A61B 5/6804; A61B 5/4312; A61B 5/4288; A61B 5/742

(57) ABSTRACT

A system for detecting changes in breast comprises a garment and a user device. The garment comprises a plurality of layers of fabric, one or more fabric-based sensors configured to detect the changes to at least one layer of fabric, a plurality of conductive nodes electrically coupled to one or more locations on a fabric-based sensor among the one or more fabric-based sensors, and a processor electrically connected to the plurality of nodes. The changes in the fabric-based sensor is detected by measuring signals between at least two nodes among the plurality of nodes. The processor is configured to obtain data measured from the at least two nodes among the plurality of nodes and transmit the data to the user device. The user device is configured to receive data from the processor and process the data.

### CROSS-REFERENCE TO RELATED APPLICATIONS

This application claims the benefit of U.S. Provisional Application No. 63/300,781 titled “SENSOR-BASED BRA TO MEASURE BREAST MILK OUTPUT AND MONITOR CHANGES IN BREAST VOLUME BEFORE AND AFTER BREASTFEEDING OR BREAST PUMPING,” filed Jan. 19, 2022, the entire contents of which is incorporated herein by reference.

### BACKGROUND

According to statistical data provided by the Centers for Disease Control and Prevention (CDC), around four out of five mothers (80%) in the U.S. start out breastfeeding, and more than half of mothers continue breastfeeding for at least six months. Breastfeeding provides various health benefits to infants and mothers. However, it's not unusual for breastfeeding mothers to encounter all kinds of challenges, such as clogged ducts, low/high milk supply, pain and depression, inconvenience of undressing, etc. Knowledge of milk production can help mothers with breastfeeding in many ways during their transition to new parenthood.

Various types of sensors have been studied for measuring milk production. Certain sensors may provide accurate readings when measuring changes of breasts related to milk production. However, those sensors may have complex structures causing difficulties in manufacturing and daily maintenance. For instance, liquid conductor-based sensors, which include conductive liquid contained in an elastomeric body, may be embedded in a garment to measure changes in breast volume. In some studies, fabric-based sensors have been investigated to monitor biophysical characteristics, such as heart rates, of mothers as well as babies. However, the information may not be sufficient to accurately indicate milk production.

Because existing technologies have these limitations, there is a need for developing techniques for monitoring milk production.

### SUMMARY

In an exemplary embodiment, the present disclosure provides a system for detecting changes in breast. The system comprises a garment and a user device. The garment comprises a plurality of layers of fabric, one or more fabric-based sensors disposed on at least one layer among the plurality of layers of fabric, a plurality of conductive nodes electrically coupled to one or more locations on a fabric-based sensor among the one or more fabric-based sensors, and a processor electrically connected to the plurality of nodes. The one or more fabric-based sensors are configured to obtain electrical measurements indicating changes to the at least one layer of fabric in the garment based on an external force or pressure applied to the at least one layer of fabric. The fabric-based sensor is disposed on a layer among the plurality of layers of fabric. The fabric-based sensor detects the changes to the layer of fabric. The detection of the changes by the fabric-based sensor is obtained by measuring signals between at least two nodes among the plurality of nodes. The processor is configured to obtain data measured from the at least two nodes among the plurality of nodes and transmit the data to a user device. The user device is configured to receive data from the processor and process the data.

In a further exemplary embodiment, the one or more fabric-based sensors comprise a stretch sensor disposed on a layer of fabric. The stretch sensor measures stretch in the layer of fabric caused by an external force.

In a further exemplary embodiment, the one or more fabric-based sensors comprise a pressure sensor disposed on a layer of fabric. The pressure sensor measures pressure applied to the layer of fabric by an external force.

In a further exemplary embodiment, the signals measured from the at least two nodes among the plurality of nodes are related to changes in resistance corresponding to a portion of the fabric-based sensor between the at least two nodes among the plurality of nodes.

In a further exemplary embodiment, the user device is further configured to cause display of information obtained by processing the data, and wherein the information is associated with breastfeeding.

In a further exemplary embodiment, the information comprises one or more of: a quantity of milk produced, a quantity of milk stored in breasts, a quantity of milk expressed, changes in milk volume production over short intervals, changes in milk volume production over long intervals, rate of milk production over short intervals, rate of milk production over long intervals, irregularities in milk production, irregularities in milk output, irregularities in duration of nursing or breasting pumping, and irregularities in schedule of nursing or breast pumping.

In a further exemplary embodiment, the user device is further configured to receive user inputs. The user inputs comprises one or more of: a daily diet, a baby's sleeping schedule, a baby's weight, changes in a baby's weight, the user's sleeping schedule, illness of the baby, illness of the user, the user's daily work schedule, and the user's mental health status.

In a further exemplary embodiment, the user device is further configured to provide medical advice or treatment.

In a further exemplary embodiment, the user device is further configured to provide a social media platform for a user to signup. The user provides information and receives advice on the social media platform.

In a further exemplary embodiment, the garment further comprises one or more massaging pads. The processor is further configured to cause the one or more massaging pads to operate.

In a further exemplary embodiment, the processor causes the one or more massaging pads to operate in response to receiving instructions via user inputs or determining changes associated with breasts based on the data.

In a further exemplary embodiment, the processor is electrically connected to the plurality of nodes via at least one of conductive fabric, conductive tapes, wires, conductive threads, and a flexible printed circuit board (PCB).

In a further exemplary embodiment, the one or more fabric-based sensors and the processor are detachable from the smart bra..

In a further exemplary embodiment, the garment further comprises a top clip and a bottom clip, wherein the plurality of layers of fabric comprises four layers. An outer layer among the four layers provides modesty and is detachable via the top clip. An inner camisole layer among the four layers comprises a slit and is detachable via the bottom clip. A top inner layer and a bottom inner layer among the four layers rest behind the inner camisole layer and are detachable via the bottom clip. The top inner layer overlaps the bottom inner layer, and the top inner layer and the bottom inner layer form a natural opening.

In a further exemplary embodiment, the garment is a bra, a tank top or other garment.

In another exemplary embodiment, the present disclosure provides a garment. The garment comprises a plurality of layers of fabric, one or more fabric-based sensors disposed on at least one layer among the plurality of layers of fabric, a plurality of conductive nodes electrically coupled to one or more locations on a fabric-based sensor among the one or more fabric-based sensors, and a processor electrically connected to the plurality of nodes. The one or more fabric-based sensors are configured to obtain electrical measurements indicating changes to the at least one layer of fabric in the garment based on an external force or pressure applied to the at least one layer of fabric. The fabric-based sensor is disposed on a layer among the plurality of layers of fabric. The fabric-based sensor detects the changes to the layer of fabric. The detection of the changes by the fabric-based sensor is obtained by measuring signals between at least two nodes among the plurality of nodes. The processor is configured to obtain data measured from the at least two nodes among the plurality of nodes and transmit the data to a user device.

In a further exemplary embodiment, the one or more fabric-based sensors comprise a stretch sensor disposed on a layer of fabric. The stretch sensor measures stretch in the layer of fabric caused by an external force.

In a further exemplary embodiment, the one or more fabric-based sensors comprise a pressure sensor disposed on a layer of fabric. The pressure sensor measures pressure applied to the layer of fabric by an external force.

In a further exemplary embodiment, the signals measured from the at least two nodes among the plurality of nodes are related to changes in resistance corresponding to a portion of the fabric-based sensor between the at least two nodes among the plurality of nodes.

In yet another exemplary embodiment, the present disclosure provides a method for detecting changes in breast. The method comprises measuring signals between at least two nodes among a plurality of nodes in a garment to obtain data indicating the changes in beast from a fabric-based sensor in the garment, and transmitting the data to a user device. The garment comprises a plurality of layers of fabric, one or more fabric-based sensors disposed on at least one layer among the plurality of layers of fabric, a plurality of conductive nodes electrically coupled to one or more locations on a fabric-based sensor among the one or more fabric-based sensors, and a processor electrically connected to the plurality of nodes. The one or more fabric-based sensors are configured to obtain electrical measurements indicating changes to the at least one layer of fabric in the garment based on an external force or pressure applied to the at least one layer of fabric. The fabric-based sensor is disposed on a layer among the plurality of layers of fabric. The fabric-based sensor detects the changes to the layer of fabric. The detection of the changes by the fabric-based sensor is obtained by measuring signals between at least two nodes among the plurality of nodes. The processor is configured to obtain data measured from the at least two nodes among the plurality of nodes and transmit the data to a user device.

### BRIEF DESCRIPTION OF DRAWINGS

The present systems and methods are described in detail below with reference to the attached drawing figures, wherein:

FIG. 1A is a block diagram of a smart garment implementing various functions, in accordance with some embodiments.

FIG. 1B illustrates multiple layers in an exemplary garment used as a smart garment as demonstrated in FIG. 1A, in accordance with some embodiments.

FIGS. 1C-1E illustrate front, back, and inside views of the exemplary garment in FIG. 1B, respectively.

FIG. 2 is a flowchart of a method for detecting changes in breast volume by using a smart garment, in accordance with some embodiments.

FIGS. 3A-3F demonstrate various exemplary configurations of sensors and conductive nodes in a garment, in accordance with some embodiments.

FIG. 4 is a block diagram of exemplary functions provided by a user device, to facilitate interactions with a smart garment as shown in FIG. 1A , in accordance with some embodiments.

FIG. 5 demonstrates an exemplary garment with massaging capabilities, in accordance with some embodiments.

FIG. 6 is a system block diagram demonstrating an exemplary smart system for milk tracking and breast massaging, in accordance with some embodiments.

### DETAILED DESCRIPTION

Systems are disclosed related to a smart garment that is comfortable as an all-day wearable and capable of monitoring changes in breast (e.g., volume, weight, shape, etc.), thereby providing in-depth information (e.g., milk production) to help mothers with breastfeeding/pumping.

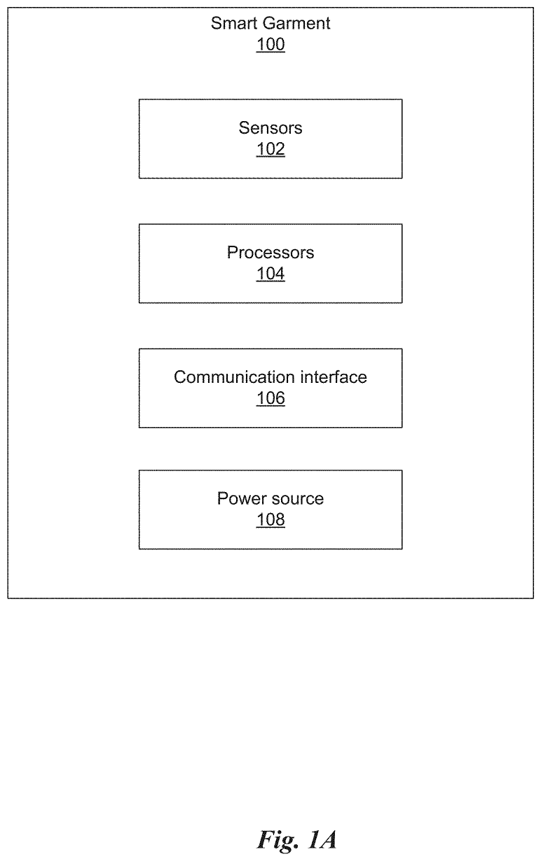

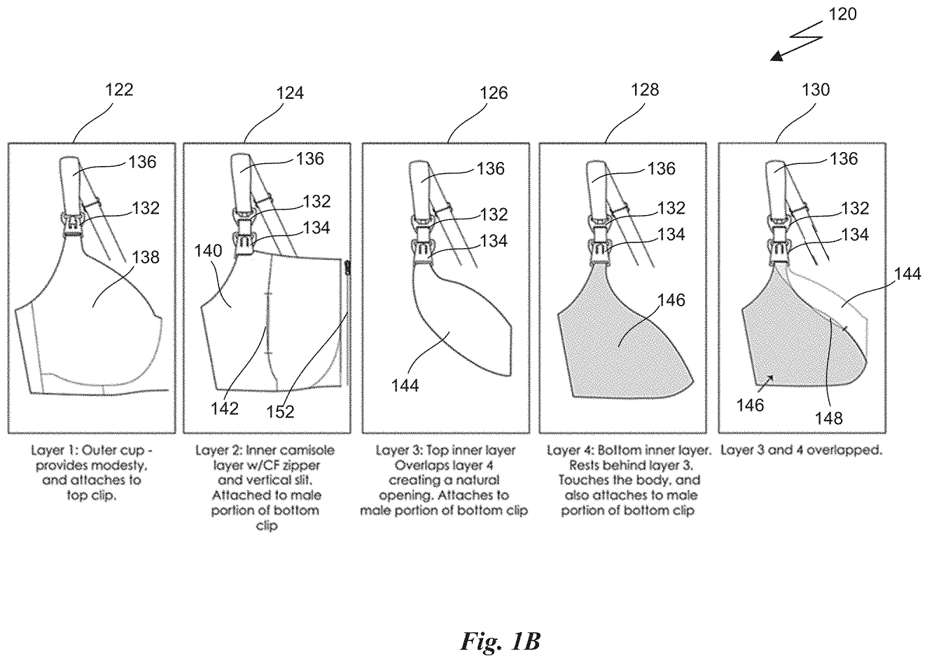

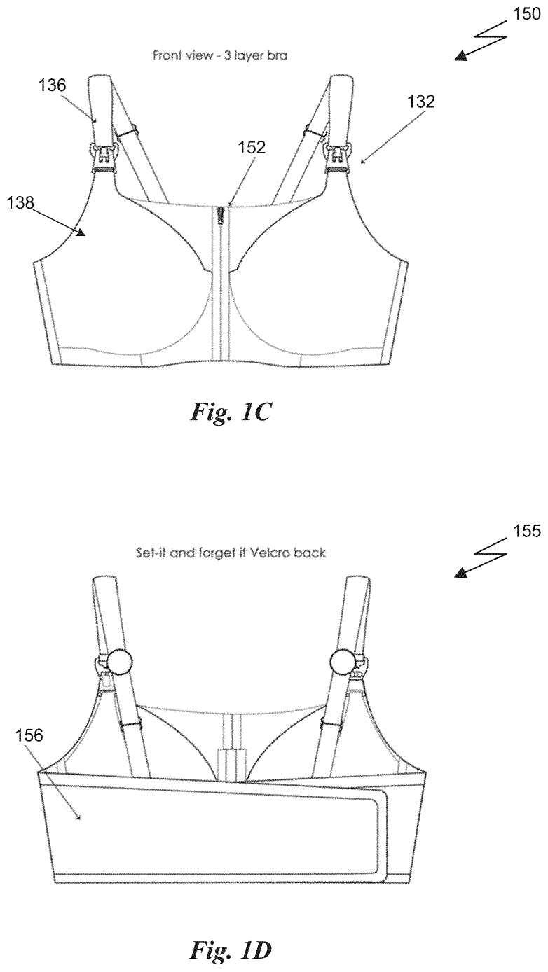

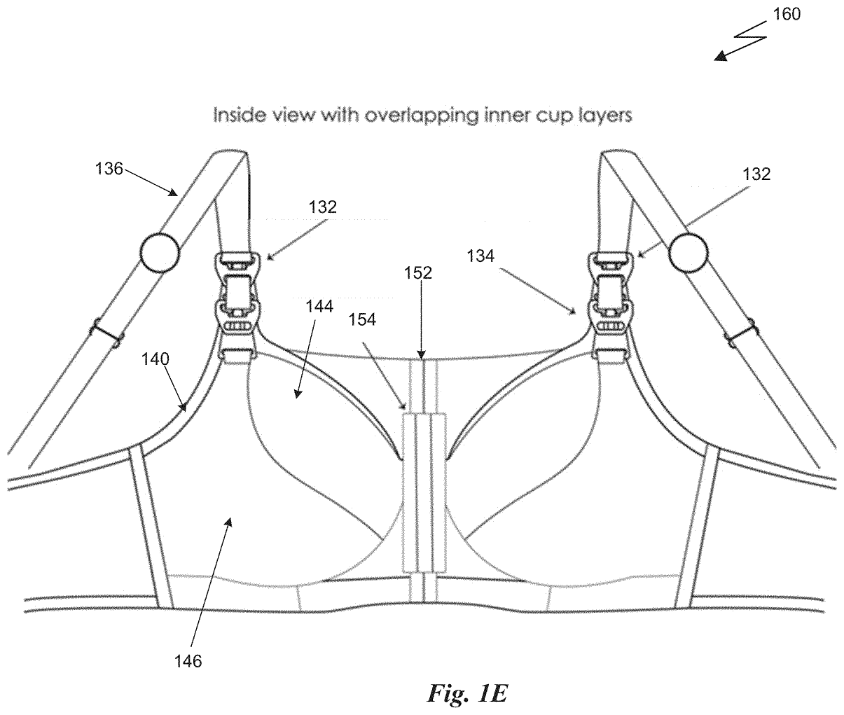

FIG. 1A is a block diagram of a smart garment 100 implementing various functions, in accordance with some embodiments. The smart garment 100 may be designed to be a comfortable all-day wearable (e.g., a bra, a tank top, etc.), which is nursing-friendly and compatible with a broad spectrum of breast-pump devices. As shown in FIG. 1A, the smart garment 100 may include various components, such as one or more sensors 102, one or more processors 104, a communication interface 106, a power source 108, and other suitable components, to facilitate monitoring of changes in breast, such as changes in shape, size, volume, etc.

In some examples, the sensors 102 may be made of sensing fabrics, which may exhibit changes in electrical characteristics subject to an external force. The sensing fabrics may include conductive and non-conductive fibers with different types of constructions (e.g., knitted or woven). When applying pressure on a specific point of the sensing fabrics, the deformation of the sensing fabrics may cause changes in electrical characteristics, such as surface resistance, thereby measuring changes in compression, stretch or tension in the sensing fabrics. In the present disclosure, a stretch sensor is referred to as a sensor that measures stretch in the sensing fabrics, and a pressure sensor is referred to as a sensor that measures pressure applied to the sensing fabrics. Examples of the fabric-based sensors include a pressure sensor made from cotton, polyester with stainless steel fibers in a knitted construction, another pressure sensor made from pure cotton with stainless steel fibers in a knitted construction, and a stretch sensor made from carbon and polyester fibers in a woven construction. A number of conductive nodes may be electrically coupled to multiple locations on a sensor 102. The changes of resistance in the sensor 102 may be obtained by performing electrical measurements, such as voltage-current measurements, between node pairs. A measurement between a pair of nodes may capture changes in resistance in the portion of the sensor 102 between the pair of nodes. Therefore, the configuration of the sensor 102, such as the size, shape, orientation, and position, as well as the positions of the conductive nodes may affect the performance of the sensor 102. The conductive nodes on the sensor 102 may be electrically connected to the one or more processors 104, which controls data acquisition from the sensor 102. It will be noted that other types of sensors (e.g., temperature sensors, humidity sensors, etc.) may be used as sensors 102 or in combination with the sensors 102 described in the present disclosure to provide information from other perspectives.

The processors 104 may be configured to perform measurements according to measurement settings. The settings may include selections of node pairs, voltage/current/frequency values, and/or other suitable parameters. A processor 104 can be a controller, a microcontroller, a central processing unit (CPU), a processing chip, and any types of processing components. The processors 104 may be integrated on a chip or in a module, which may further include peripheral circuits to process the raw data from the sensors 102. For instance, the peripheral circuit may include diodes, filters, amplifiers, and other types of electrical components, to amplify signals and suppress noises. In some variations, the processors 104 may perform certain calculations, such as summation, averaging, and subtraction, on the raw data from the sensors 102. In some examples, the smart garment 100 may include a housing space for the processors 104, which may support attachment and detachment of the processors 100 therefrom.

The communication interface 106 in the smart garment 100 may be configured to receive data from and transmit data to a user device, which may further analyze the data to provide further information, for example, information related to breastfeeding/pumping. The communication interface 106 may include WiFi, Bluetooth, or other types of wireless communication chipsets, to provide a data communication connection. In such an implementation, the communication interface 106 can send and receive electrical, electromagnetic or optical signals that carry digital/analog data streams representing various types of information via a network. The network can typically include a cellular communication network, a Wireless Local Area Network (“WLAN”), a Wide Area Network (“WAN”), a personal area network (PAN), or the like. The user device may be various types of devices/systems with computational power. For example, the user device may be embodied as a smartphone, a personal computer, or a server.

The power source 108 provides power to the circuit formed by the sensors 102, the processors 104, the communication interface 106, and the power source 108. The power source 108 may include non-rechargeable batteries, rechargeable batteries with charging circuits or circuits designed to receive power from an external power source.

FIG. 1B illustrates multiple layers in a garment 120 as an embodiment of the smart garment 100 as shown in FIG. 1A, in accordance with some embodiments. The garment 120 is embodied as a bra for the purpose of demonstration, which is not intended to limit the scope of the present disclosure.

As illustrated in FIG. 1B, the bra 120 may include four layers, referred to as Layer 1, Layer 2, Layer 3, and Layer 4, respectively. Blocks 122, 124, 126, 128, and 130 demonstrate the design of the four layers in one side of the bra 120, which is mirrored in the other side.

As shown in block 122, Layer 1 may include an outer cup 138, which is attached to a top clip 132 on a strap 136 and provides modesty to a user. As shown in block 124, Layer 2 may include an inner camisole layer 140 beneath the outer cup 138 in Layer 1. Certain designs of Layer 2 provide convenient access to inner layers of the bra 120, such as the attachment of Layer 2 to a bottom clip 134 on the strap 136 and the vertical slit designed on each side of the cups. As shown in block 124, the bra 120 may further include a zipper 152 (e.g., a CF zipper) located at the center of the bra 120, through which the bra 120 may be fully open. Blocks 126 and 128 present Layer 3 and Layer 4 in separate views, whereas block 130 provides a combined view of Layer 3 and Layer 4. Layer 3 may include a top inner layer 144, which may be attached to the bottom clip 134. Layer 4 may include a bottom inner layer 146, which may also be attached to the bottom clip 134. Layer 4 may rest behind Layer 3 and touch the body of the user. Layer 3 and Layer 4 may be partially overlapped in such a manner that a natural opening 148 may be formed by these two layers.

By way of implementing the design of four layers as shown in FIG. 1B, a smart garment 100 can be compatible with direct nursing, as well as all standard and wearable breast pumps. The breast pumps may be electrical or non-electrical. As such, the smart garment 100 may be worn as an all-day wearable, which allows a mother to breastfeed/pump without taking off and putting on the garment before and after each instance of breastfeeding/pumping. Furthermore, the smart garment 100 may provide optimal support to maximizing contact with breasts, which provides comfort to the user as well as enhance the sensitivity of sensors 102.

FIGS. 1C-1E are front, back, and inside views 150, 155, and 160 of the bra 120 as shown in FIG. 1B, respectively. FIG. 1C shows the front view 150 of Layer 1 including outer cups 138, a zipper 152 in Layer 2, top clips 132 and straps 136. In some examples, the straps 136 may be padded straps for increased comfort. The outer cups 138 may be released via the top clips 132. FIG. 1D provides the back view 155 of the bra 120, which adopts a design of back Velcro wings 156. As shown in FIG. 1E, the inside view 160 provides more insights of the inner layers, including Layer 2 140, Layer 3 144, and Layer 4 146. Layer 2 may also include a pocket 154 placed behind the zipper 152, which may be a pleated pocket used to accommodate the processors 104. Layer 2 140, Layer 3 144, and Layer 4 146 may all be attached to the bottom clips 134 on the straps 136.

The following provides further detail of certain features in the bra 120 as shown in FIG. 1B-1E.

Padded straps 136. As the breast gets full and empty with milk, additional stress and weight on the shoulders occurs. Thin straps would cause for a lot of the weight to stretch out the straps as opposed to the bra cup, so while the main driver is padded straps for comfort, thicker padded straps help to localize the stretch changes to the bra cup so that the sensors 102 can accurately measure the changes in stretch/compression of the bra material in the breast cup area.

Clips for outer and inner layers, such as top clips 132 and bottom clips 134. Mothers typically breastfeed and breast pump between one to eight times every day. Therefore, it is crucial to mitigate the amount of time and effort required to ‘setup’ the breast pump while pumping and ‘position the baby easily’ while breastfeeding. As such, top clips 132 are provided to the front layer of the bra 120 to ‘release’ the outer cup(s) for easy access to the breast pumping layers, including Layer 2, Layer 3 and Layer 4 with slits/openings. A second pair of bottom clips 134 are provided to the breast pumping layers to ‘release’ them so as to have unobstructed access to the nipple while breastfeeding a baby.

Zipper 152 in the front of the bra 120. Most nursing bras today have a fastening mechanism in the back which makes it difficult for moms to put on the bra. A sleek zipper fastener in the front of the bra 120 makes it easy to put on and take off the bra 120. Further, since the bra 120 has multiple layers, adding a zipper 152 in the front of the bra 120 would provide rigid support to ‘bind’ the layers and keep them in place, which helps with reducing noises while collecting data from the sensors 102 throughout the day.

Velcro fastener in the back of the bra 120. A new mother's body goes through a lot of changes throughout the first year after giving birth. An adjustable fastener is added in the back of the bra 120, to make the bra 120 highly adjustable in order to conform to changing bodies of mothers. FIG. 1D illustrates an exemplary embodiment of Velcro wings 156. The wide band at the back would also provide good structure to the bra 120 and keep it close to the body at all time, ensuring good contact between the breasts and the sensors 102 in the bra 120 to achieve accurate readings from the sensors 102.

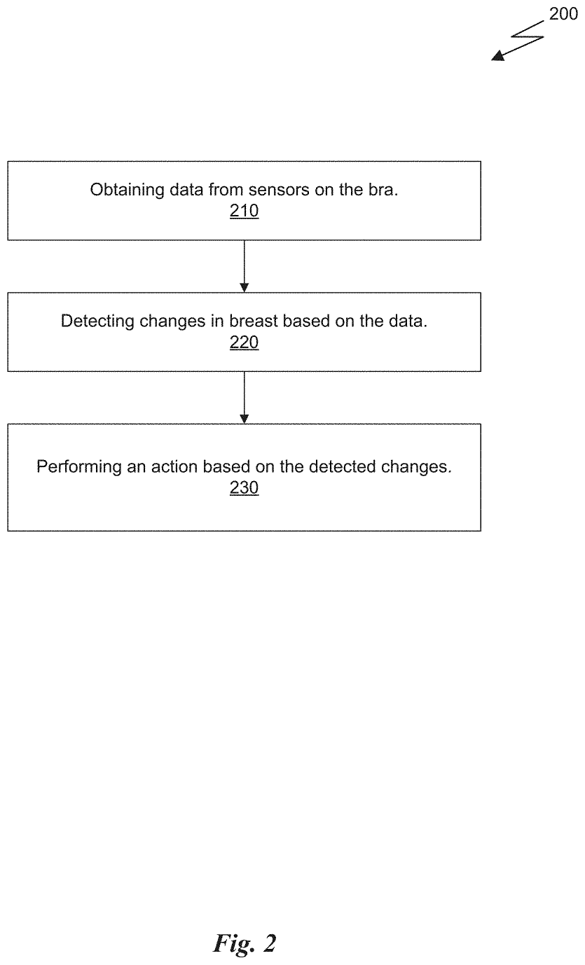

FIG. 2 is a flowchart of a method 200 for detecting changes in breast volume by using a smart garment 100 as shown in FIG. 1A, in accordance with some embodiments. The smart garment 100 may implement the multi-layer design as shown in the bra 120 in FIGS. 1B-1E, or other suitable designs to facilitate various functions in the present disclosure. It will be recognized that the method 200 may be performed in any suitable environment and that any of the blocks in FIG. 2 may be performed in any suitable order.

In block 210, the smart garment 100 may obtain data from the sensors 102. The smart garment 100 may include a plurality of layers of fabric, such as the four layers in the bra 120 as shown in FIG. 1B. The sensors 102 may be disposed on at least one layer among the plurality of layers of fabric, which may exhibit changes in electrical characteristics (e.g., in resistance) subject to an external force. In some instances, the sensors 102 may be fabric-based sensors, which may be attached to a layer in the smart garment 100. Alternatively, the sensors 102 may be made as part of a layer in the smart garment 100. In some variations, the sensors 102 may include a stretch sensor disposed on a layer of fabric in the smart garment 100, which may measure stretch in the layer of fabric caused by a tension force. In some examples, the sensors 102 may include a pressure sensor disposed on a layer of fabric in the smart garment 100, which may measure pressure in the layer of fabric caused by a compression force. It will be recognized that both stretch and pressure sensors may be disposed on a layer of fabric in the smart garment 100, such that stretch and pressure in the layer of fabric can be measured concurrently at different locations in the layer of fabric.

The changes in the sensors 102 may be measured from a plurality of conductive nodes that are electrically coupled to the sensors 102. The conductive nodes may be integrated on a layer of fabric in the smart garment 100 and electrically connected to the sensors 102 via conductive materials, such as conductive fabric, tapes, wires, threads, or flexible printed circuit board (FPCB) that is embedded in at least one layer of the fabric. In some variations, the conductive nodes may directly contact the sensors 102. The sensor 102 may deform at different rates at different locations as the volume of breast changes. The processors 104 may select different pairs of conductive nodes to detect changes in resistance between the nodes, such that the smart garment 100 may be used to monitor changes of breasts at different locations. Examples of changes in breast to be monitored include but not limited to shape, size, volume, weight, etc.

The sensors 102 may be placed in one of the multiple layers in the smart garment 100. The sensors 102 may be of various shapes and sizes in order to accurately detect changes in breast volume and breast shape in various areas. The collected data by the sensors 102 will in turn produce accurate information about milk production and milk expression over a short period (e.g., per pumping session) and longer period (e.g., daily, weekly, monthly change in milk production, duration of milk expression, milk output etc.) and other suitable information (e.g., breathing patterns and postures/movements of the user).

The sensors 102 may be made from various fabrics. In some examples, the sensors 102 may be easily detachable from the smart garment 100. In some instances, the sensors 102 may be permanently attached to one or multiple layers of fabric(s) in the smart garment 100. In some variations, the sensors 102 may be integrated directly into the fabric that forms one layer of the smart garment 100.

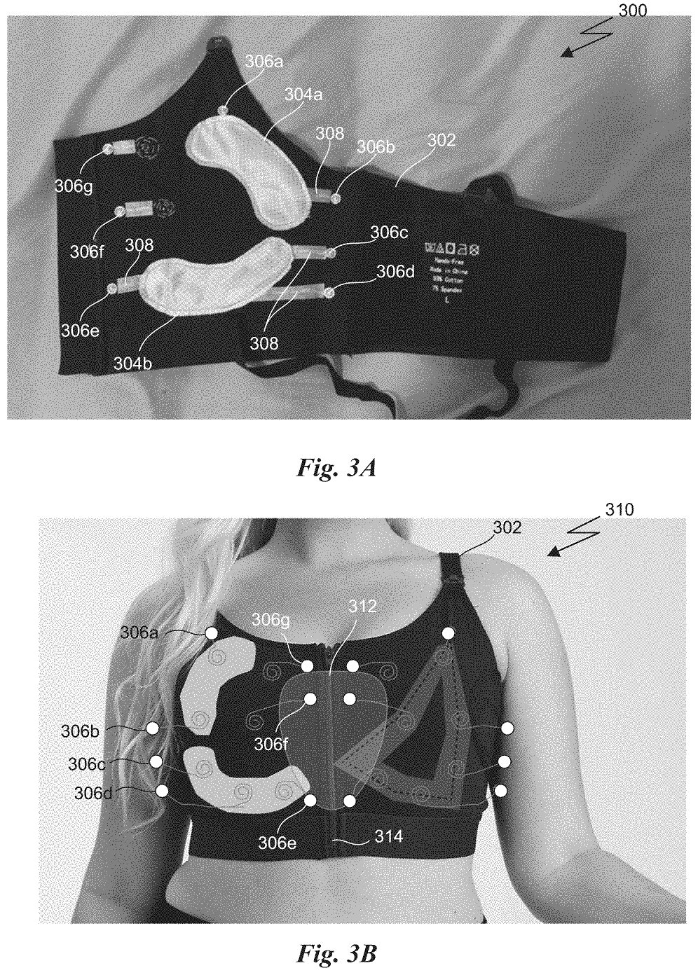

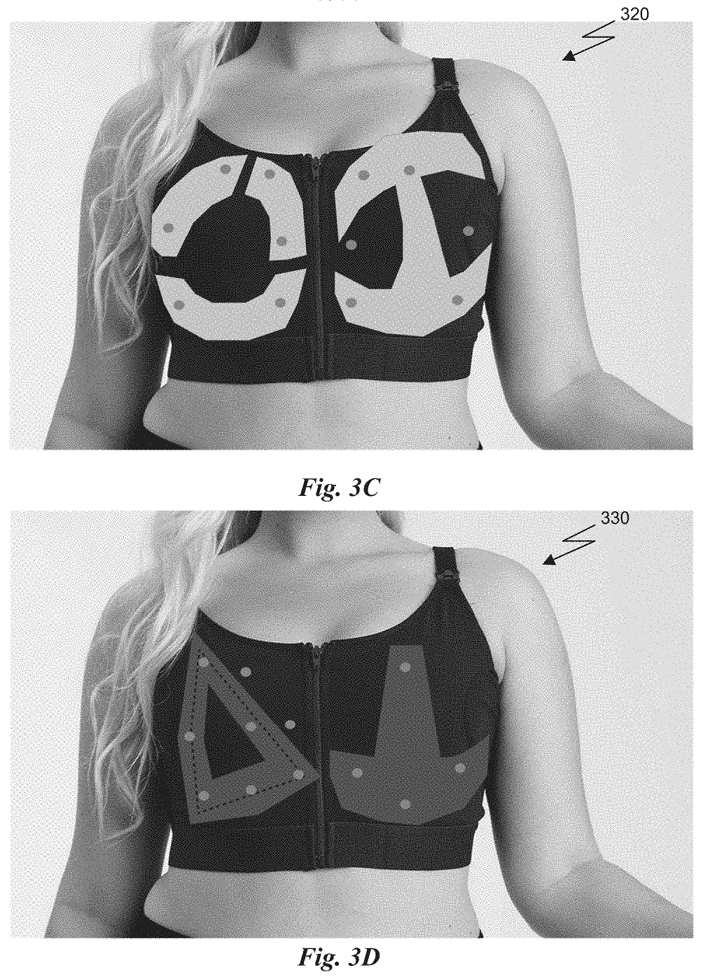

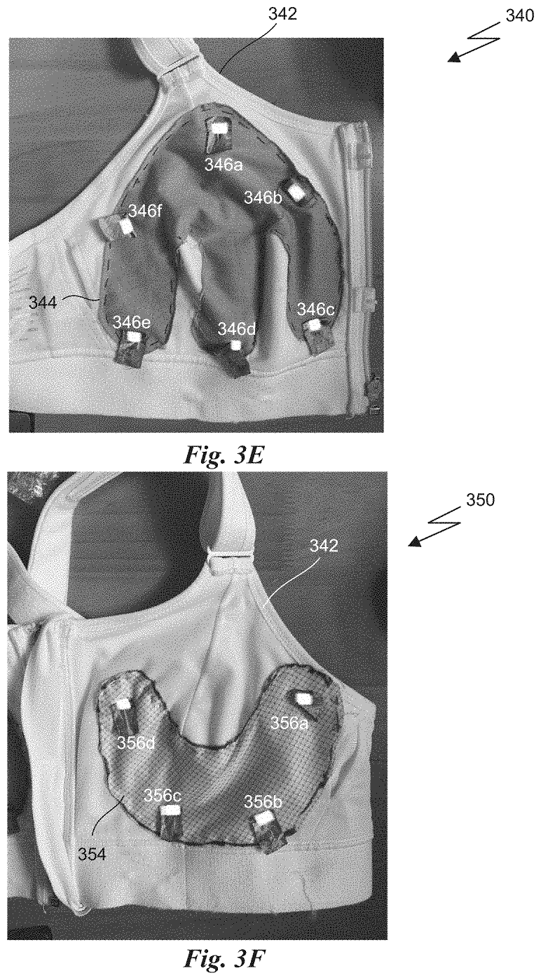

FIGS. 3A-3F demonstrate exemplary configurations of sensors and conductive nodes on an exemplary garment. The configurations demonstrate exemplary shapes/positions of the sensors and/or exemplary positions of conductive nodes. It should be noted that other configurations of the sensors and conductive nodes may be implemented in the smart garment 100 for data collection.

In a configuration 300 as shown in FIG. 3A, two sensors 304a and 304b are placed on an inner layer of a garment 302. Seven conductive nodes are integrated on the garment 302, which are coupled to seven locations on each side of the garment 302. In this configuration 300, the conductive nodes 306a and 306b are electrically coupled to the sensor 304a via conductive threads/wires (e.g., thread 308). The conductive nodes 306c, 306d and 306e are electrically coupled to the sensor 304b via threads 308. The conductive nodes 306f and 306g are not electrically coupled to any of the sensors 304a and 304b on the garment 302. Changes in the sensor 304a may be measured from the pair of the nodes 306a and 306b. Changes in the sensor 304b may be measured from various combinations of node pairs, such as the pair of nodes 306c and 306d, the pair of nodes 306c and 306e, and the pair of nodes 306d and 306e.

The same set of conductive nodes may be used with different sensors. FIG. 3B is an embodiment 310 that demonstrates two exemplary configurations on the garment 302 with the identical set of conductive nodes 306a-306g as shown in FIG. 3A. The left side of the garment 302 illustrates the configuration 300 as shown in FIG. 3A, whereas the right side of the garment 302 illustrates a different configuration 312. The configuration 312 includes a single sensor 314, which is electrically coupled to six of the seven conductive nodes on the garment 312. The processors 104 may be placed in an area 312 beneath the zipper 314. FIGS. 3C and 3D demonstrate additional configurations with different sets of conductive nodes and different sensors.

Different types of sensors may be used in a smart garment 100, which may require different set of conductive nodes to achieve optimal sensitivity. FIGS. 3E and 3F demonstrate two different types of sensors.

FIG. 3E demonstrates a configuration 340 on a garment 342, which includes a pressure sensor 344 and six conductive nodes 346a-346f. The sensor 344 in this configuration is designed with “gaps” cut out between node pairs to allow for easier conformity of the sensor 344 around the breast cup and hence reduce noise due to creases and seams. Since the “weight” of the breast exerts force downwards, the pressure sensor 344 between these six nodes would likely be most sensitive to detect the changes in weight of the breast due to milk production (and extraction). FIG. 3F demonstrates a configuration 350 including a stretch sensor 354 and four conductive nodes 356a-356d. Since the volume of the breast is concentrated in the “cup” size, the stretch sensor 344 between these four nodes would likely be most sensitive to changes in cup sizes and hence changes in volume of the breast due to milk production (and extraction).

The processors 104 may be configured to store measurement settings, such as voltage/current values, a sampling rate (i.e., frequency), node identifications, etc., and perform measurements according to the settings. In some instances, the processors 104 may perform simple calculations, such as summation, averaging, and subtraction, on the data obtained from the sensors 102. Furthermore, the processors 104 may transmit the data to a user device via a communication interface 106. The user device may be various types of devices/systems capable of performing computational tasks. For example, the user device may be embodied as a smartphone, a smart watch, a tablet, a personal computer, a server, and any other suitable types of devices/systems.

In block 220, the user device may process the received data by applying various algorithms to determine changes in breast. In some examples, the node pairs to be measured are first calibrated. The calibration may provide a relationship between the electrical characteristics of each node pair and the physical characteristics (e.g., the deformation) in the portion of the sensor 102, which may be translated to changes in breast (e.g., breast shape/size) at a location corresponding to the node pair. To this end, the user device may determine changes in breast based on the measured data and the calibration results, which may be translated to other information associated with breastfeeding/pumping. In some instances, the user device may track changes of the data collected by the sensors 102, interpret the changes, discover patterns based on the changes, and/or determine specific/abnormal events (clogging, leaking, etc.). The user device may identify the specific/abnormal events based on historical data of the user and/or statistical data of peer users.

In some variations, the user device may provide various functions including visually presenting the obtained information in a user interface (UI) and interacting with the user, by executing software programs using one or more processors in the user device. The software programs may be provided by an application (APP) installed on the user device and stored in a memory integrated therein. The memory may be any non-transitory type of mass storage, such as a read-only memory (“ROM”), a flash memory, a dynamic random-access memory (“RAM”), and/or a static RAM.

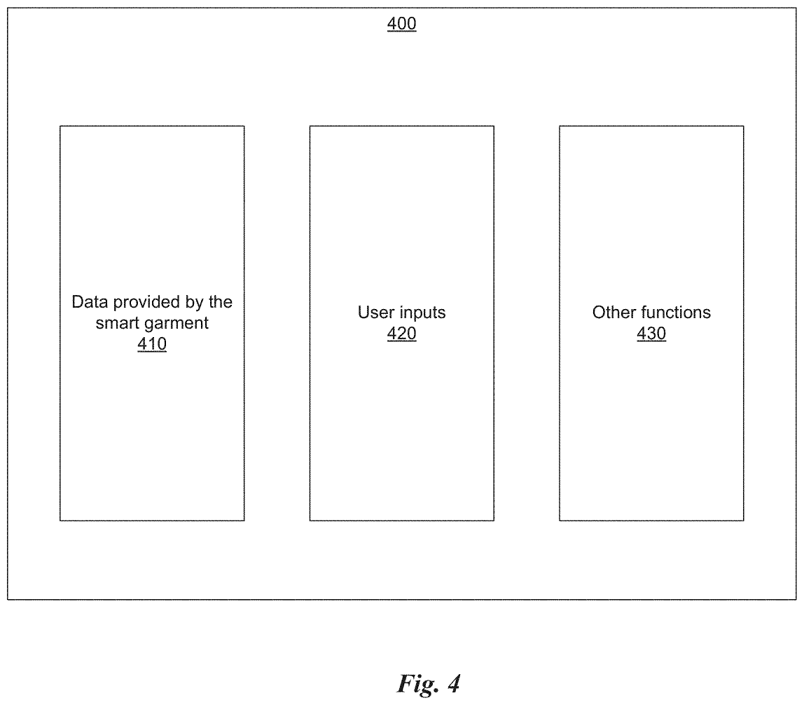

FIG. 4 is a block diagram of exemplary functions provided by a user device 400, to facilitate interactions with a smart garment 100 as shown in FIG. 1A. As shown in FIG. 4, the user device 400 may provide functions 410 related to processing data provided by the smart garment 100, functions 420 related to interactions with a user, and other functions 430. The user device 400 may further include a communication interface configured to communicate with the smart garment 100 via a network. Additionally, the communication interface may include various I/O devices such as a keyboard, a mouse, a touchpad, a touch screen, a microphone, a camera, a biosensor, etc. A user may input data to the user device 400 (e.g., a mobile device) through the communication interface. In some examples, the user device 400 may include a display or be communicatively coupled to a display device, on which the user device 400 may present a UI for user inputs and data depiction.

In some instances, the user device 400 may perform the functions 410 to process the data received from the smart garment 100 to obtain information associated with breastfeeding/pumping and cause display of the information in a UI. The information may include a quantity of milk produced/stored in breasts, a quantity of milk expressed, changes in milk volume production over short intervals (e.g., during an event of breastfeeding/pumping) or long intervals (e.g., days, weeks, months, etc.), rate of milk production over short/long intervals, irregularities in milk production/output, irregularities in duration of breastfeeding or pumping, irregularities in schedule of breastfeeding or pumping, and any other suitable information.

In some variations, the user device 400 may perform the functions 420 to receive user inputs via I/O devices. The user device 400 may cause display of preferences, which could include default values or inputs by the user, in the UI. Examples of the user inputs/preferences may include a daily diet, a baby's sleeping schedule, a baby's weight, changes in a baby's weight, the user's sleeping schedule, illness of the baby/user, the user's daily work schedule, the user's mental health status, and any other suitable information. In some variations, the user device 400 may determine default values for certain preferences based on patterns discovered from the sensor data. The user device 400 may determine measurement settings (e.g., selection of node pairs) based on the user inputs. The user may be able to manually select the node pair(s) and/or measurement settings via options provided by the user device 400.

The user device 400 may provide other functions 430 to the user. The other functions 430 may involve an action to be performed by the user device 400 or the smart garment 100, which will be described in detail in block 230.

In block 230, an action may be performed by the smart garment 100 and/or the user device 400 based on the changes in breast .

In some examples, the user device 400 may track changes in breast and detect specific events by performing the functions 410. Subsequently, the user device 400 may cause display of the information related to the changes in breast in a UI. In a further example, the user device 400 may generate notifications for the detected specific events by performing the functions 430. The notifications may present information related to the specific events in texts, graphs, symbols, and other suitable forms.

In some instances, the user device 400 may determine a measurement setting based on the user inputs processed by the functions 420 and transmit corresponding instructions to the smart garment 100. Once receiving the instructions, the processors 104 in the smart garment 100 may perform measurements according to the instructions.

In some variations, the user device 400 may receive data backed insights and help from medical experts on lactation, sleep, mental health via an APP installed on the user device 400. For instance, such information may be available in a database, which may be sorted by the user device 400 based on the data collected from the smart garment 100 and the information extracted by performing the functions 410. The user device 400 may determine helpful information via the APP to address stress/anxiety during breastfeeding, stress/anxiety due to change of schedule, stress/anxiety due to milk supply issues, stress/anxiety due to baby's health, stress/anxiety due to work balance after maternity leave, etc. In some examples, the APP may provide a secured and HIPAA (short for “The Health Insurance Portability and Accountability Act”) compliant “communication” interface between users and medical health experts to facilitate medical advice and treatment. Such advice and/or treatment may include and not be limited to the following: lactation consultation from certified professionals, mental health consultations from certified professionals, sleep consultation from experts, diet consultation from experts, etc. In this example, the app may provide the medical health experts access to information about specific users collected from the users and/or computer by the milk tracking algorithm, to facilitate personalized, data driven treatment.

In some instances, the user device 400 may provide a social media platform for a user to signup by performing functions 430, through which the user may share “crowdsourced” information with or receive advice from other mothers. The platform may have communities/groups of users based on specific criteria to provide easier, more efficient means of communications among the users. Such communities may include and not be limited to the following: communities based on geographical location, communities based on term of labor, communities based on age of baby/stage of breastfeeding, communities based on specific kinds of complications faced by users (e.g., clogged ducts, undersupply of breastmilk, mastitis, oversupply of breastmilk, baby's latching, mommy thumb, etc.). As an example, the social media platform may include a community platform for expecting and new mothers to discuss their postpartum journeys, crowdsource answers to lactation and other prenatal/postpartum related questions, tip and tricks from other mothers and/or experts etc.

In some examples, the smart garment 100 may include massage pads. The processors 104 may be further configured to cause the massage pads to operate at different modes. The user device 400 may perform the functions 430 to control the massage pads and/or monitor the operation of the massage pads. The operation of the massage pads may be controlled via user inputs. Alternatively, the user device 400 may determine an operation of the massage pads in response to detection of certain changes in breast or detection of pressure/stretch between certain node pairs. The user may be able to change the settings related to the massage pads via the functions 420/430 provided by the user device 400. In some variations the massage functions (e.g., the massage pads) may be combined or co-exist with the sensors 102 in the smart garment 100.

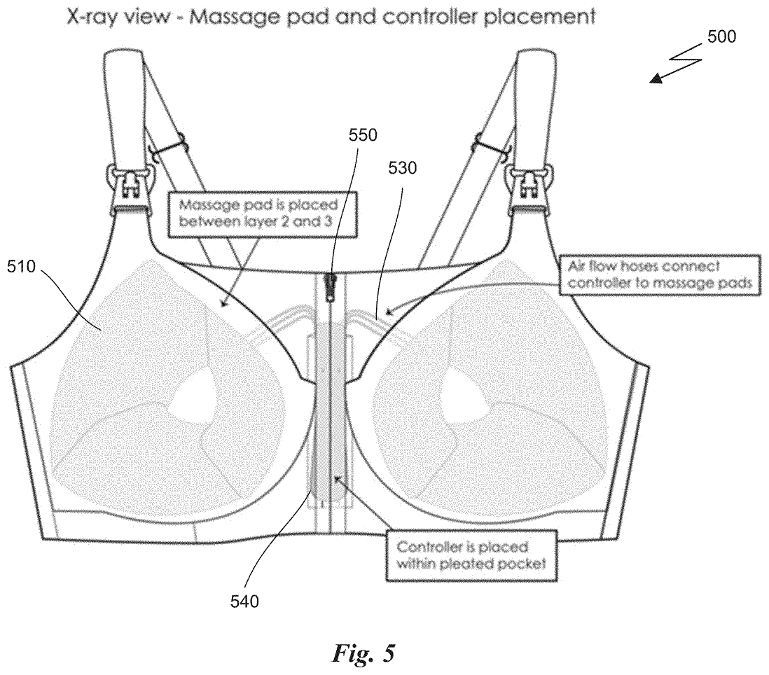

FIG. 5 demonstrates an exemplary garment 500 with massaging capabilities, in accordance with some embodiments. The smart garment 500 may include the components in the smart garment 100 as demonstrated in FIG. 1A, and have multiple layers as shown in FIG. 1B.

As shown in FIG. 5, a set of massage pads 510 may be placed in each side of the smart garment 500. Each of the massage pads 510 may be separately controlled by the processors 104 in the smart garment 500. In this example, the massage pads 510 may be placed between Layer 2 and Layer 3 as shown in blocks 124 and 126 in FIG. 1B. The processors 104 and the power source 108 may be placed within a pleated pocket 540 located in the middle of the smart garment 500. The pleated pocket 540 may be accessed via a zipper 550. The processors 104 may also house pneumatic/hydraulic pumps and pneumatic/hydraulic valves in order to enable massage functionality in the smart garment 500 using breast pads (or massage cushions) (described in U.S. patent Ser. No. 15/598,050, the entire contents of which is incorporated herein by reference). The massage pads 510 and the processors 104 in the pleated packet 540 may be connected by air flow hoses 530. By ways of implementing the massage capabilities, the smart garment 500 doubles up as a compression/massage garment with milk tracking capabilities. In some examples, the smart garment 500 may provide a closed loop feedback/control system for further massage and compression personalization.

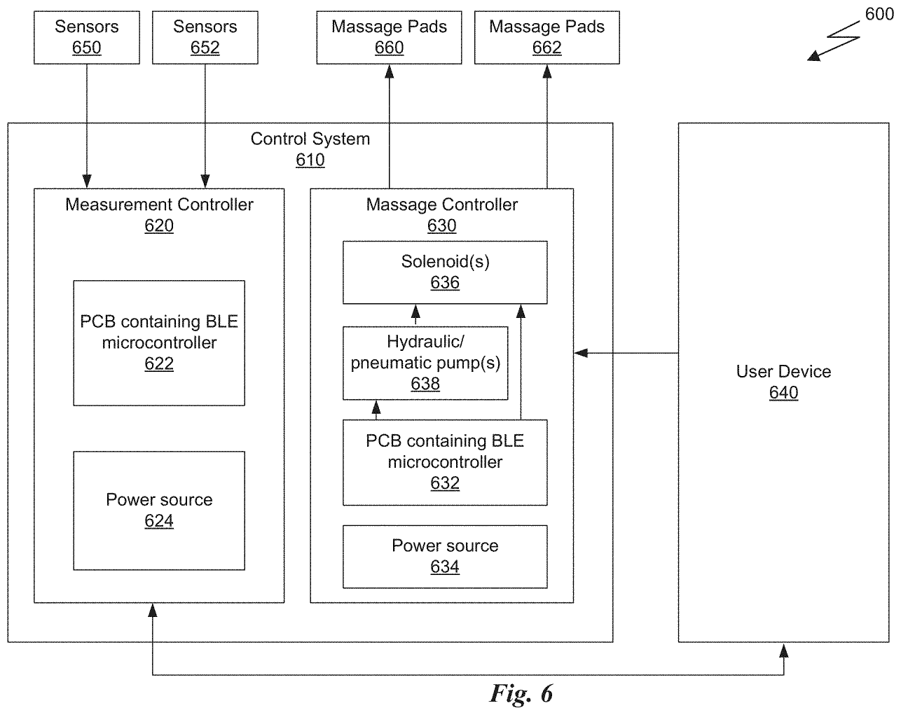

FIG. 6 is a system block diagram demonstrating an exemplary smart system 600 for milk tracking and breast massaging, in accordance with some embodiments.

The smart system 600 may include a smart garment and a user device 640. The smart garment may house one or more sensors 650 and one or more massage pads 660 in one side of the garment, and one or more sensors 652 and one or more massage pads 662 in the other side of the garment. Additionally, the smart garment may house a control system 610, which may include a measurement controller 620 and a massage controller 630. The measurement controller 620 may include a PCB 622 integrated with Bluetooth connectivity (BLE) and a microcontroller and a power source 624 to power the electrical components in the smart garment. The Bluetooth connectivity may be configured to transmit data to the user device 640. The microcontroller may be configured to control measurements performed on the sensors 650 and 652.

The massage controller 630 may include one or more solenoids 636, one or more hydraulic/ pneumatic pumps 638, a PCB 632 integrated with BLE and a microcontroller, and a power source 634. The microcontroller may work with the solenoid(s) 636 and the hydraulic/pneumatic pump(s) 638 to control the massage pads 660 and 662. In some variations, the control system 610 may include a single PCB 622, which is configured to control both the measurements on the sensors and the operations of the massage pads. The control system 610 may include a single power source 624 to supply power to all the components in the smart garment.

The user device 640 may provide various functions as shown in FIG. 4, which may be facilitated by executing corresponding software programs stored in a memory by using one or more processors in the user device 640. The user device 640 may process the data collected by the sensors 650 and 652 and cause display of various information in a UI. Furthermore, the user may be able to control the measurements on the sensors and the operations of the massage pads in the smart garment via the functions provided by the user device 640. In some examples, the user data may be stored in the cloud, and/or the user device may connect to cloud servers for certain functions as described in the present disclosure.

It is noted that the techniques described herein may be embodied in executable instructions stored in a computer readable medium for use by or in connection with a processor-based instruction execution machine, system, apparatus, or device. It will be appreciated by those skilled in the art that, for some embodiments, various types of computer-readable media can be included for storing data. As used herein, a “computer-readable medium” includes one or more of any suitable media for storing the executable instructions of a computer program such that the instruction execution machine, system, apparatus, or device may read (or fetch) the instructions from the computer-readable medium and execute the instructions for carrying out the described embodiments. Suitable storage formats include one or more of an electronic, magnetic, optical, and electromagnetic format. A non-exhaustive list of conventional exemplary computer-readable medium includes: a portable computer diskette; a random-access memory (RAM); a read-only memory (ROM); an erasable programmable read only memory (EPROM); a flash memory device; and optical storage devices, including a portable compact disc (CD), a portable digital video disc (DVD), and the like.

It should be understood that the arrangement of components illustrated in the attached Figures are for illustrative purposes and that other arrangements are possible. For example, one or more of the elements described herein may be realized, in whole or in part, as an electronic hardware component. ther elements may be implemented in software, hardware, or a combination of software and hardware. Moreover, some or all of these other elements may be combined, some may be omitted altogether, and additional components may be added while still achieving the functionality described herein. Thus, the subject matter described herein may be embodied in many different variations, and all such variations are contemplated to be within the scope of the claims.

To facilitate an understanding of the subject matter described herein, many aspects are described in terms of sequences of actions. It will be recognized by those skilled in the art that the various actions may be performed by specialized circuits or circuitry, by program instructions being executed by one or more processors, or by a combination of both. The description herein of any sequence of actions is not intended to imply that the specific order described for performing that sequence must be followed. All methods described herein may be performed in any suitable order unless otherwise indicated herein or otherwise clearly contradicted by context.

The use of the terms “a” and “an” and “the” and similar references in the context of describing the subject matter (particularly in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. Furthermore, the foregoing description is for the purpose of illustration only, and not for the purpose of limitation, as the scope of protection sought is defined by the claims as set forth hereinafter together with any equivalents thereof. The use of any and all examples, or exemplary language (e.g., “such as”) provided herein, is intended merely to better illustrate the subject matter and does not pose a limitation on the scope of the subject matter unless otherwise claimed. The use of the term “based on” and other like phrases indicating a condition for bringing about a result, both in the claims and in the written description, is not intended to foreclose any other conditions that bring about that result. No language in the specification should be construed as indicating any non-claimed element as essential to the practice of the invention as claimed.

What is claimed is:

1. A system for detecting changes in breast, comprising:
a garment, comprising:
a plurality of layers of fabric;
one or more fabric-based sensors disposed on at least one layer among the plurality of layers of fabric, the one or more fabric-based sensors configured to obtain electrical measurements indicating changes to the at least one layer of fabric in the garment based on an external force or pressure applied to the at least one layer of fabric;
a plurality of conductive nodes electrically coupled to one or more locations on a fabric-based sensor among the one or more fabric-based sensors, the fabric-based sensor disposed on a layer among the plurality of layers of fabric, wherein the fabric-based sensor detects the changes to the layer of fabric, and wherein the detection of the changes by the fabric-based sensor is obtained by measuring signals between at least two nodes among the plurality of nodes; and
a processor electrically connected to the plurality of nodes, the processor configured to obtain data measured from the at least two nodes among the plurality of nodes and transmit the data to a user device; and
the user device configured to receive data from the processor and process the data.

2. The system of claim 1, wherein the one or more fabric-based sensors comprise a stretch sensor disposed on a layer of fabric, wherein the stretch sensor measures stretch in the layer of fabric caused by an external force.

3. The system of claim 1, wherein the one or more fabric-based sensors comprise a pressure sensor disposed on a layer of fabric, wherein the pressure sensor measures pressure applied to the layer of fabric by an external force.

4. The system of claim 1, wherein the signals measured from the at least two nodes among the plurality of nodes are related to changes in resistance corresponding to a portion of the fabric-based sensor between the at least two nodes among the plurality of nodes.

5. The system of claim 1, wherein the user device is further configured to cause display of information obtained by processing the data, and wherein the information is associated with breastfeeding.

6. The system of claim 5, wherein the information comprises one or more of:
a quantity of milk produced;
a quantity of milk stored in breasts;
a quantity of milk expressed;
changes in milk volume production over short intervals;
changes in milk volume production over long intervals;
rate of milk production over short intervals;
rate of milk production over long intervals;
irregularities in milk production;
irregularities in milk output;
irregularities in duration of nursing or breasting pumping; and
irregularities in schedule of nursing or breast pumping.

7. The system of claim 1, wherein the user device is further configured to receive user inputs, and the user inputs comprises one or more of:
a daily diet;
a baby's sleeping schedule;
a baby's weight;
changes in a baby's weight;
the user's sleeping schedule;
illness of the baby;
illness of the user;
the user's daily work schedule; and
the user's mental health status.

8. The system of claim 1, wherein the user device is further configured to provide medical advice or treatment.

9. The system of claim 1, wherein the user device is further configured to provide a social media platform for a user to signup, and wherein the user provides information and receives advice on the social media platform.

10. The system of claim 1, wherein the garment further comprises one or more massaging pads, and wherein the processor is further configured to cause the one or more massaging pads to operate.

11. The system of claim 10, wherein the processor causes the one or more massaging pads to operate in response to receiving instructions via user inputs or determining changes associated with breasts based on the data.

12. The system of claim 1, wherein the processor is electrically connected to the plurality of nodes via at least one of conductive fabric, conductive tapes, wires, conductive threads, and a flexible printed circuit board (PCB).

13. The system of claim 1, wherein the one or more fabric-based sensors and the processor are detachable from the smart bra.

14. The system of claim 1, wherein the garment further comprises a top clip and a bottom clip, wherein the plurality of layers of fabric comprises four layers, and
wherein:
an outer layer among the four layers provides modesty and is detachable via the top clip,
an inner camisole layer among the four layers comprises a slit and is detachable via the bottom clip,
a top inner layer and a bottom inner layer among the four layers rest behind the inner camisole layer and are detachable via the bottom clip, and
the top inner layer overlaps the bottom inner layer, and the top inner layer and the bottom inner layer form a natural opening.

15. The system of claim 1, wherein the garment is a bra, a tank top or other garment.

16. A garment, comprising:
a plurality of layers of fabric;
one or more fabric-based sensors disposed on at least one layer among the plurality of layers of fabric, the one or more fabric-based sensors configured to obtain electrical measurements indicating changes to the at least one layer of fabric in the garment based on an external force or pressure applied to the at least one layer of fabric;
a plurality of conductive nodes electrically coupled to one or more locations on a fabric-based sensor among the one or more fabric-based sensors, the fabric-based sensor disposed on a layer among the plurality of layers of fabric, wherein the fabric-based sensor detects the changes to the layer of fabric, and wherein the detection of the changes by the fabric-based sensor is obtained by measuring signals between at least two nodes among the plurality of nodes; and
a processor electrically connected to the plurality of nodes, the processor configured to obtain data measured from the at least two nodes among the plurality of nodes and transmit the data to a user device.

17. The garment of claim 16, wherein the one or more fabric-based sensors comprise a stretch sensor disposed on a layer of fabric, wherein the stretch sensor measures stretch in the layer of fabric caused by an external force.

18. The garment of claim 16, wherein the one or more fabric-based sensors comprise a pressure sensor disposed on a layer of fabric, wherein the pressure sensor measures pressure applied to the layer of fabric by an external force.

19. The garment of claim 16, wherein the signals measured from the at least two nodes among the plurality of nodes are related to changes in resistance corresponding to a portion of the fabric-based sensor between the at least two nodes among the plurality of nodes.

20. A method for detecting changes in breast, comprising:
measuring signals between at least two nodes among a plurality of nodes in a garment, to obtain data indicating the changes in beast from a fabric-based sensor in the garment; and
transmitting the data to a user device,
wherein the garment comprises:
a plurality of layers of fabric;
one or more fabric-based sensors comprising the respective fabric-based sensor, the one or more fabric-based sensors disposed on at least one layer among the plurality of layers of fabric, the one or more fabric-based sensors configured to obtain electrical measurements indicating changes to the at least one layer of fabric in the garment based on an external force or pressure applied to the at least one layer of fabric;
the plurality of conductive nodes electrically coupled to one or more locations on the respective fabric-based sensor among the one or more fabric-based sensors, the respective fabric-based sensor disposed on a layer among the plurality of layers of fabric,
wherein the respective fabric-based sensor detects the changes to the layer of fabric, and
wherein the detection of the changes by the respective fabric-based sensor is obtained by measuring signals between the at least two nodes among the plurality of nodes; and
a processor electrically connected to the plurality of nodes, the processor configured to obtain data measured from the at least two nodes among the plurality of nodes and transmit the data to the user device.
